(19) United States
(12) Patent Application Publication
(10) Pub. No.: US 2020/0188466 A1
(43) Pub. Date: Jun. 18, 2020

(54) COMPOSITIONS OF SCHISANDRA EXTRACTS AND METHODS THEREOF

(71) Applicant: Reyoung Corporation, Gathersburg, MD (US)

(72) Inventors: Yushan ZHAO, Potomac, MD (US); Dezu MIAO, Yiyuan County (CN); Shujie HOU, Gaithersburg, MD (US); Jingshan HUANG, Yiyuan County (CN)

(73) Assignee: Reyoung Corporation, Gaithersburg, MD (US)

(21) Appl. No.: 16/607,842

(22) PCT Filed: Apr. 4, 2018
(86) PCT No.: PCT/US2018/026035

§ 371 Date: Oct. 24, 2019

Related U.S. Application Data
(60) Provisional application No. 62/489,573, filed on Apr. 25, 2017.

(51) Int. Cl. A61K 36/79; A61K 31/09; A61K 31/36; A61K 31/365; A61K 31/165; A61K 31/19; A61K 47/00; A61K 36/752; A61K 31/496; A61K 31/4196; A61K 31/4164; A61K 31/7048

(52) U.S. Cl. CPC A61K 36/79; A61K 31/09; A61K 31/36; A61K 31/365; A61K 31/165; A61K 31/7048; A61K 47/00; A61K 36/752; A61K 31/496; A61K 31/4196; A61K 31/4164; A61K 31/19

(57) ABSTRACT

This disclosure is directed to compositions comprising the Schisandra Sphenanthera extract and a plant-based compound that comprises one or more of triptolide, colchicine, wilforlide A, celastrol, and their analogs or derivatives. Further disclosed herein are methods of increasing the bioavailability of these plant-based compounds and methods of treating diseases with the compositions.

### CROSS-REFERENCE TO RELATED PATENT APPLICATIONS

This application claims priority under 35 U.S.C. § 119(e) to U.S. Provisional Application No. 62/489,573, filed Apr. 25, 2017, the content of which is hereby incorporated by reference in its entirety.

### BACKGROUND

Triptolide (“TPL”) is a bioactive compound originally isolated from the plant Tripterygium wilfordii Hook F (“TWHF”). Studies show that triptolide and its derivatives have a broad spectrum of bioactivities, e.g., anti-inflammation, immunomodulation, antiproliferation, proapoptosis, and neuroprotection. Triptolides are used or implicated in treating a number of diseases or medical conditions, including autoimmune diseases, transplantation rejection, cancers, infertility, and other diseases. Qiu et al., Drugs R D., 4(1):1-18 (2003). In particular, the anti-tumor effects of triptolide are at least partially related to its functions in inhibition of cell growth and metastasis, apoptosis and improving chemoradiosensitivity during cancer therapy. Triptolide has been approved for Phase I clinical trials for treating prostate cancer. Meng et al., Chin J Cancer Res., 26(5): 622-626 (2014). Triptolide can also function as a potent tumor angiogenesis inhibitor. He et al., Int. Journal of Cancer, 126, 266-278 (2010).

Colchicine is another plant-based compound, which was originally identified from Colchicum autumnale (autumn crocus, meadow saffron), Gloriosa superba (glory lily), and other plants. Colchicine is well recognized as an effective therapy for gout, familial Mediterranean fever (FMF), and Behçet's disease. Schwartz et al., Semin Arthritis Rheum; 29(5):286-95 (2000). Colchicine is also used for treating inflammatory disorders prone to fibrosis and was proposed as an effective therapy for cardiovascular diseases. Verma et al., BMC Cardiovascular Disorders, 15:96 (2015).

However, the therapeutic efficacies of triptolide and colchicine are affected by toxicity and low bioavailability during administration. Moreover, plant-based compounds or herbal drugs (e.g., triptolide and colchicine) are often associated with poor absorption rates. Studies also show that triptolide can lead to hepatotoxicity and reproductive toxicity, e.g., by decreasing sperm or azoospermia in males and menstrual quantity or amenorrhoea in females. Zheng et al., CNS Neuroscience & Therapeutics, 19: 76-82 (2013). Toxicity caused by colchicine can lead to gastrointestinal upset and organ dysfunction.

Thus, the undesirable features associated with plant-based compounds (e.g., triptolide and colchicine) highlight the need to develop a new composition with reduced or eliminated toxicity and enhanced bioavailability.

### SUMMARY OF THE INVENTION

The disclosure provides a pharmaceutical composition which comprises, alternatively consists essentially of, or yet further consists of an extract from Schisandra sphenanthera and a plant-based compound. In one embodiment, the extract from Schisandra sphenanthera comprises, alternatively consists essentially of, or yet further consists of a compound isolated from Schisandra sphenanthera. In another embodiment, the compound isolated from Schisandra sphenanthera comprises, alternatively consists essentially of, or yet further consists of Schisandrin A, Schisandrin B, Schisandrin C, Schizandrol A, Schizandrol B, Schisantherin A, or the combination thereof. In another embodiment, the plant-based compound comprises, alternatively consists essentially of, or yet further consists of one or more of triptolide, colchicine, wilforlide A, celastrol, and their derivatives or analogs. In another embodiment, the triptolide analogs comprise one or more of 16-hydroxy-triptolide, triptonide, and tripdiolide.

In another aspect, the disclosure relates to methods of increasing bioavailability of a plant-based compound in a subject by administering to the subject a therapeutically effective amount of a pharmaceutical composition comprising a Schisandra sphenanthera extract. In another embodiment, the extract from Schisandra sphenanthera comprises, alternatively consists essentially of, or yet further consists of Schisandrin A, Schisandrin B, Schisandrin C, Schizandrol A, Schizandrol B, Schisantherin A, or a combination thereof. In another embodiment, the pharmaceutical composition comprises inhibitors of cytochrome P enzymes. In another embodiment, the pharmaceutical composition comprises inhibitors of P-glycoprotein. In another embodiment, the plant-based compound comprises, alternatively consists essentially of, or yet further consists of one or more of triptolide, colchicine, wilforlide A, celastrol, and their derivatives or analogs. In another embodiment, the triptolide analogs comprise one or more of 16-hydroxy-triptolide, triptonide, and tripdiolide.

In another aspect, the disclosure relates to methods of treating and/or preventing a disease in a subject, comprising, alternatively consisting of, or yet further consisting of administering to the subject an effective amount of a pharmaceutical composition, wherein said pharmaceutical composition comprises, alternatively consists essentially of, or yet further consists of an extract from Schisandra sphenanthera and a plant-based compound. In another embodiment, the extract from Schisandra sphenanthera comprises, alternatively consists essentially of, or yet further consists of Schisandrin A, Schisandrin B, Schisandrin C, Schizandrol A, Schizandrol B, Schisantherin A, or a combination thereof. In another embodiment, the plant-based compound comprises, alternatively consists essentially of, or yet further consists of one or more of triptolide, colchicine, wilforlide A, celastrol, and their derivatives or analogs. In another embodiment, the triptolide analogs comprise one or more of 16-hydroxy-triptolide, triptonide, and tripdiolide.

### BRIEF DESCRIPTION OF THE DRAWINGS

FIG. 1 shows the structures of triptolide and its analogs.

FIG. 2 shows the structure of colchicine.

FIG. 3 shows the structures of celastrol and wilforlide A.

FIG. 4 shows the structures of compounds isolated from Schisandra sphenanthera.

FIG. 5 shows plasma triptolide concentration-time profiles.

FIG. 6 shows plasma celastrol concentration-time profiles.

FIG. 7 shows plasma colchicine concentration-time profiles.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof, as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting the invention. As used herein, the singular forms “a,” “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

The term “comprising” or “comprises” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amounts of other ingredients and substantial method steps recited. Embodiments defined by each of these transition terms are within the scope of this invention.

As used here, the term “plant-based compound” refers to a compound that is isolated, extracted, purified, or derived from a plant. The term includes both natural and non-natural product and may also include compound that is not isolated from a plant but has a similar or same structure. In one embodiment, the plant-based compound includes a chemical that is synthesized but has a same or similar structure with a compound that is extracted or isolated from a plant. In some embodiments, the plant-based compound includes analogs or derivatives that are similar to an original compound isolated from a plant, but differ in composition and may or may not have some or all of the activities of the original compound. In one embodiment, the analogs and derivatives are naturally occurring or non-naturally occurring compounds. Non-limiting examples of plant-based compounds of this disclosure include triptolides, colchicines, glycosides (e.g., cardiac glycoside, cyanogenic glycoside, glucosinolate, saponin, and anthraquinone glycoside), wilforlide A, celastrol, flavonoids, proanthocyanidins, tannins, terpenoids (e.g., monoterpenoids, sesquiterpenoids, and phenylpropanoids), diterpenoids, resins, lignans, pyrrolizidine alkaloids, tropane alkaloids, alkaloids, furocoumarins, naphthodianthrones, and their derivatives and analogs.

The term “extract” or “plant extract,” as used herein interchangeably, refers to a substance in any form that is extracted, either individually or in a group, from or similar to any part or parts of a plant or plant material. In one embodiment, the plant extract comprises as a substance that is synthesized but has a same or similar structure with the substance that is extracted from a plant. Examples of parts of plants include, but are not limited to, leaves, flowers, roots, seeds, pods, stems, fruits, seed coats, and buds. In some embodiments, the plant extract exists in any form, including, but not limited to, liquid, gas, or, solid. In some embodiments, the plant extract is a compound.

As used herein, the term “bioavailability” is defined as the relative amount of a drug administered in a pharmaceutical product that enters the systemic circulation in an unchanged form, and the rate at which this occurs. See Principles of Clinical Pharmacology edited by Atkinson et al. (Academic Press, 2001) at page 33. One of ordinary skill in the art appreciates that the bioavailability of a drug can be determined by measuring parameters influencing absorption and elimination of the drug, and that these parameters are well known in the art and more fully explained in Principles of Clinical Pharmacology edited by Atkinson et al. (Academic Press, 2001). Parameters for measuring drug absorption include, but are not limited to the maximum drug concentration in plasma (Cₘₐₓ), the time needed to reach this maximum (Tₘₐₓ), and the area under the plasma or serum-concentration-vs.-time curve (AUC₀₋₄) after administration of the drug. Parameters often used to assess elimination of the drug include, but are not limited to the terminal elimination half time (T_{1/2}), defined as the time required for half of an administered drug dose to be eliminated, and the mean residence times (MRT₀₋₄), defined as the average time the drug is accessible during all passages through the system before being irreversibly cleared.

The terms “dosage” or “dosage regiment” is defined herein as the amount needed for effectiveness of each of the various disease states. Dosage regimens may be adjusted to provide the optimum desired response (e.g., a therapeutic or prophylactic response). For example, a single dosage may be administered or several divided doses may be administered over time or the dose may be proportionally reduced or increased as indicated by the exigencies of the therapeutic situation. Dosage unit form refers to physically discrete units such as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals. In some embodiments, the dosage of a particular compound is provided as absolute weight. In some embodiment, the dosage of a particular compound is provided as mass ratio, wherein the mass ratio is the fraction of a particular compound out of the total composition. In some embodiments, the dosage is provided as mg compound per kg total bodyweight of the subject to whom the composition is provided, and this dosage format is hereinafter designated mg/kg. In some embodiments, the dosage is provided in hourly, daily, weekly, or monthly dosage regimens.

The terms “patient,” “subject,” “individual,” and the like are used interchangeably herein and refer to any animal, or cells thereof, whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g., horse, cow, pig, goat, or sheep). In especially preferred embodiments, the patient, subject, or individual is a human.

The terms “disease” or “disorder” and the like are used interchangeably herein and refer to conditions that impair normal tissue function. A person of ordinary skill in the art understand that diseases or disorders can be caused by genetic abnormalities, by aging (when the problem is cause by time dependent deterioration of tissue), or by contracting outside agents such as toxins or infectious agents. A person of ordinary skill in the art will understand that diseases have primary causes and secondary symptoms. Hence, a treatment can target the underlying cause of the disease or the treatment can alleviate the secondary symptoms of the disease. Non-limiting examples of diseases and disorders include autoimmune diseases, neurodegenerative disease, transplantation rejection, cancers, infertility, gout, familial Mediterranean fever, cardiovascular diseases, and Behçet's disease. Non-limiting examples of cancer include pancreatic cancer, renal cancer, small cell lung cancer, brain cancer, neural cancer, bone cancer, lymphoma, colon cancer, uterine cancer, breast cancer, leukemia, liver cancer, prostate cancer, skin cancer, and melanoma.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, treatment of a cancer includes, but is not limited to, elimination of the cancer or the condition caused by the cancer, remission of the tumor, inhibition of the cancer, and reduction or elimination of at least one symptom of the tumor.

The term “administering” or “administration” of an agent to a subject includes any route of introducing or delivering to a subject a compound to perform its intended function. A route of administration is the path by which a drug, fluid, poison, or other substance is taken into the body. Routes of administration are generally classified by the location at which the substance is applied. Administration can be carried out by any suitable route, including orally, intranasally, parenterally (intravenously, intramuscularly, intraperitoneally, or subcutaneously), intramuscularly, by inhalation, or topically. Administration includes self-administration and the administration by another.

The phrase “concurrently administering” refers to administration of at least two agents to a patient over a period of time. Concurrent administration includes, without limitation, separate, sequential, and simultaneous administration.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the complete administration of one of the active ingredients before administration of the other or others commences. It is thus possible to administer one of the active ingredients over several minutes, hours, or days before administering the other active ingredient or ingredients.

The term “simultaneous administration” refers to the administration of at least two ingredients by the same route and at the same time or at substantially the same time.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect. For example, an effective amount of a composition may be an amount sufficient to treat, control, alleviate, or improve the conditions related to parasitic diseases. The therapeutically effective amount of the agent may vary depending on the pathogen being treated and its severity as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

The term “triptolide” refers to a triptolide compound, a triptolide derivative or analog, a suitable homolog, or a portion thereof, capable of promoting at least one of the biological responses normally associated with triptolide. In one embodiment, the triptolide is synthesized or isolated from a natural product. In some embodiments, the triptolide of this disclosure also includes triptolide prodrugs. Non-limiting examples of triptolide prodrugs are disclosed in European Patent No. EP2427467, which is incorporated by reference in its entirety. Non-limiting examples of triptolide analogs include 16-hydroxy-triptolide, triptonide, and tripdiolide.

As used herein, the term “colchicine” refers to a colchicine compound, a colchicine derivative or analog, a suitable homolog, or a portion thereof, capable of promoting at least one of the biological responses normally associated with colchicine. In one embodiment, the colchicine is synthesized or isolated from a natural product.

As used herein, the term “wilforlide A” refers to a wilforlide A compound, a wilforlide A derivative or analog, a suitable homolog, or a portion thereof, capable of promoting at least one of the biological responses normally associated with wilforlide A. In one embodiment, the wilforlide A is synthesized or isolated from a natural product.

As used herein, the term “celastrol” refers to a celastrol compound, a celastrol derivative or analog, a suitable homolog, or a portion thereof, capable of promoting at least one of the biological responses normally associated with celastrol. In one embodiment, the celastrol is synthesized or isolated from a natural product.

The terms “isolated” and “purified” can be used interchangeably. In some embodiments, the term “isolated” can be used to refer to the extract being removed from the natural chemical environment.

The term “analog” refers to a compound in which one or more individual atoms or functional groups have been replaced, either with a different atom or a different functional, generally giving rise to a compound with similar properties. In another embodiment, the analog refers to a structure that is similar to another but differs in one or more components.

The term “derivative” refers to a compound that is formed from a similar, precursor compound by attaching another molecule or atom to the beginning compound. Further, derivatives, according to the invention, encompass one or more compounds formed from a precursor compound through addition of one or more atoms or molecules or through combining two or more precursor compounds.

The term “pharmaceutically acceptable carrier” refers to a carrier that is conventionally used in the art to facilitate the storage, administration, and/or the healing effect of a biologically active agent.

The term “pharmaceutically acceptable salts” includes herein derivatives of a plant-based compound wherein the plant-based compound is modified by making acid or base addition salts thereof, and further refers to pharmaceutically acceptable solvates, including hydrates, and co-crystals of such compounds and such salts. Examples of pharmaceutically acceptable salts include, but are not limited to, mineral or organic acid addition salts of basic residues such as amines; alkali or organic addition salts of acidic residues, and the like, as well as combinations comprising one or more of the foregoing salts. The pharmaceutically acceptable salts include non-toxic salts and the quaternary ammonium salts of the plant-based compounds. For example, non-toxic acid salts include those derived from inorganic acids such as hydrochloric, hydrobromic, sulfuric, sulfamic, phosphoric, nitric, and the like; other acceptable inorganic salts include metal salts such as sodium salt, potassium salt, cesium salt, and the like; and alkaline earth metal salts, such as calcium salt, magnesium salt, and the like, as well as combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH (where n is 0-4), and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparaginate, glutamate, and the like; and combinations comprising one or more of the foregoing salts. All forms of such derivatives of plant-based compounds are contemplated herein, including all crystalline, amorphous, and polymorph forms. Specific plant-based compounds salts include colchicine hydrochloride, colchicine dihydrochloride, and co-crystals, hydrates or solvates thereof.

As used here, the term “CYP” or “cytochrome P450” refers to a family of metabolic enzymes. Non-limiting examples of CYP enzymes include CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, and CYP4Z1.

As used here, the term “P-glycoprotein” or “P-gp” refers to a protein encoded by the multiple drug resistance 1 gene (MDR1), also known as the ATP-binding cassette subfamily B member 1 (ABCB1) gene. In some embodiments, P-gp acts as an ATP-dependent pump for transporting drug molecules out of cell interiors. In one embodiment, p-glycoprotein transport mechanism facilitates the reverse transport of substances, which diffuse or are transported inside the cell, back into the lumen of the intestine. In some embodiments, the p-glycoprotein, through its reverse transport system, functions to prevent bioavailability of substances, including beneficial drugs, by preventing the digested substance from entering the circulatory system.

As used here, the term “inhibitor” refers to a substrate that blocks or suppresses the activity, function, or effect of a target. In some embodiments, the target is a compound, a protein, a gene, a cell, or an agent. In some embodiments, the target is a CYP enzyme or a p-glycoprotein. In some embodiments, the inhibitor includes a compound that prevents binding of another molecule to an enzyme or molecular pump. In some embodiments, the inhibitor is a compound that causes downregulation of the enzyme or molecular pump. In one embodiment, the inhibitor functions to inhibit a CYP enzyme or p-glycoprotein. An inhibitor can be a competing or non-competing inhibitor. As used here, the term “non-competing inhibitor” refers to a type of inhibitor that binds to an enzyme or a target so that the enzyme or the target cannot bind to or act on another substrate. Thus, a substrate of an enzyme can be a competing inhibitor of a target (e.g., a CYP enzyme or p-glycoprotein). Non-limiting examples of substrates or inhibitors for CYP enzyme can be found at http://www.genemedrx.com/Cytochrome_P450_Metabolism_Table.php. Non-limiting examples of CYP enzyme inhibitor includes amiodarone, amprenavir, aprepitant, REYATAZ® (atazanavir), cimetidine, ciprofloxacin, clarithromycin, delavirdine, diltiazem, doxycycline, Echinacea, enoxacin, erythromycin, fluconazole, fluvoxamine, grapefruit juice, indinavir, itraconazole, ketoconazole, miconazole, nefazodone, nelfinavir, ritonavir, saquinavir, telithromycin, verapamil, and voriconazole. A CYP3A is one of the CYP enzymes, which is found in the liver and the intestine. Non-limiting examples of CYP3A inhibitors include ketoconazole, itraconazole, fluconazole, cimetidine, clarithromycin, erythromycin, troleandomycin, and grapefruit juice. Non-limiting examples of p-glycoprotein inhibitors include amiodarone, clarithromycin, erythromycin, ketoconazole, quinidine, saquinavir, and verapamil.

### Plant-Based Compound

Bioactive compounds produced from plant cells have been reported to have pharmacological effects in human and animals. The bioactive compounds or therapeutic agents from plants include both first and second metabolites from the plants. Some plant-based compounds have been highly valued for their health and therapeutic benefits, e.g., reduction of cardiovascular diseases, treatment of cancers, and alleviation of inflammatory responses. The clinical applications of plant-based compounds in therapeutic regimen have been tempered by low availability and toxicity. For example, tannins have been used to treat cold sores and fever blisters, chronic diarrhea, dysentery, bloody urine, painful joints, persistent coughs, and cancer. However, the hydrolysable tannins may potentially lead to toxicity effects among administered patients. The low water solubility of tannins further limits the clinical utility.

Tripterygium wilfordii (“TW”) is a plant that illustrates the functions of plant secondary metabolites as useful pharmaceutical agents, and also the difficulty of producing the plant products in practical yields. A number of compounds having immunosuppressive or other activities have been isolated from extracts of root tissues from TW, including tripterinin, 16-hydroxytriptolide, triptriolide, celastrol, tripchlorolide, triptophenolide, triptonide, tripterine, tripterygic acid, sesquiterpene alkaloids, isowilfordine, sesquiterpene esters, sesquiterpene polyol esters, phenanthrene derivatives, tripterygone, salaspermic acid, other diterpene lactone epoxide compounds, and diterpene quinones.

Among them, triptolide is a bioactive diterpenoid isolated from the traditional Chinese medicinal herb Tripterygium wilfordii Hook F (“TWHF”). Triptolide has a broad spectrum of potent bioactivities, e.g., anti-inflammatory, immunomodulatory, antiproliferative, proapoptotic, and neuroprotective activities. Ziaei et al., Avicenna J Phytomed 6(2): 149-64 (2016). It can be used to treat rheumatoid arthritis, systemic lupus erythematosus, ankylosing spondylitis and cancer. Studies show that triptolide is a broad-spectrum cancer suppressor and can induce apoptosis of a variety of cancer cells, including pancreatic cancer, renal cancer, small cell lung cancer, brain cancer, neural cancer, bone cancer, lymphoma, colon cancer, uterine cancer, breast cancer, leukemia, liver cancer, prostate cancer, skin cancer, and melanoma. It can also inhibit tumor growth and metastasis of cancer cells in vivo, including hematological cancers, malignancies and solid cancers. Further, triptolide can overcome the drug resistance of cancer cells and at the same time increase the sensitivity of cancer cells to other anti-cancer drugs. Triptolide further has synergistic effect when combining with chemotherapeutic drugs and ionizing radiation.

Currently, many studies are trying to investigate the anti-cancer mechanism of triptolide. Triptolide can inhibit the expression of heat shock protein 70 (“HSP 70”). As an inhibitor of heat shock protein response, triptolide can effectively inhibit the expression of HSP 70 genes and induce cell apoptosis. Arora et al., Plos One, 12:e0171827 (2017). Triptolide can inhibit nuclear factor kappa B (“NF-κB”). Yoshida et al., J Am Hear Assoc, 16: e007248 (2017). NF-κB not only promotes cancer cell proliferation but also activates oncogene and anti-apoptotic genes, which lowers the sensitivity of cancer cells towards apoptosis. In one hand, triptolide inhibits the combination of NF-κB and a specific DNA sequence at the target gene and further interferes with the transcription activity of NF-κB. On the other hand, triptolide can prevent nuclear kinase from performing phosphorylation on NF-κB trans-activating region or interfere with the nuclear accumulation of auxiliary protein of NF-κB, e.g. cAMP response element binding protein, as well as interfere with the interaction between P65 and RNA polymerase and further inhibits the transcription activity of NF-κB to promote apoptosis. In addition to the above mechanism, triptolide further exhibits its anti-cancer effect through various ways such as inhibiting ubiquitin-proteasome, affecting the activity of RNA polymerase, affecting the expression of p53 gene, activating caspase, etc.

The clinical utilities of triptolide are, however, limited due to its toxicities to multiple organs, insolubility in water, and poor bioavailability. Accumulated clinical studies disclose that hepatotoxicity and reproductive toxicity (such as decreasing sperm or azoospermia in males, and decreasing menstrual quantity or amenorrhoea in females) are two of the main toxicities caused by triptolide or medicines including triptolide. Biological studies showed that many physiological pathways are affected by the hepatotoxicity caused by triptolide, such as decreasing mitochondrial membrane potential, decreasing the protein expression of Nrf2 and its target genes, decreasing the levels of GSH, increasing ROS levels, the excessive apoptosis of hepatocytes and lipid peroxidation. Meanwhile, a recent study shows that the slow development of oocytes at different developmental stages is also related to reproductive toxicity in females caused by triptolide.

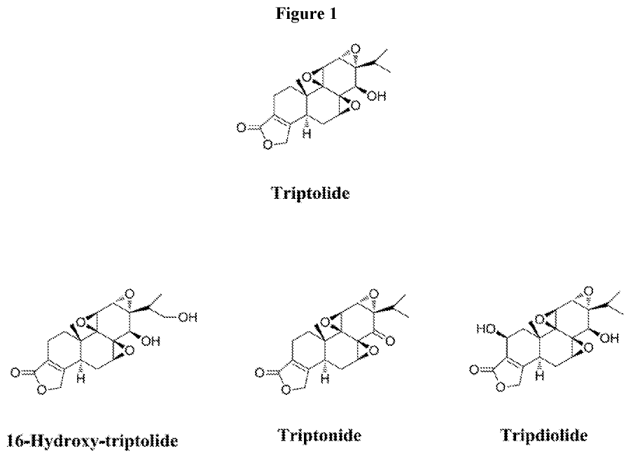

The chemical structure of triptolide and its analogs are shown in FIG. 1 and all such forms of triptolide and its pharmaceutically acceptable salts are contemplated herein, including hydrates, and co-crystals of such compounds and such salts. Examples of pharmaceutically acceptable salts include, but are not limited to, mineral or organic acid addition salts of basic residues such as amines; alkali or organic addition salts of acidic residues, and the like, and combinations comprising one or more of the foregoing salts. The pharmaceutically acceptable salts include non-toxic salts and the quaternary ammonium salts of the plant-based compounds. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH (where n is 0-4), and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparaginate, glutamate, and the like; and combinations comprising one or more of the foregoing salts. Specific triptolide salts include triptolide hydrochloride, triptolide dihydrochloride, and co-crystals, hydrates or solvates thereof.

In some embodiments, the low bioavailability of triptolide (including its analogs) is associated with metabolization by CYP3A. Pretreatment of animals with CYP3A inhibitors or inducers could significantly alter the metabolic profile of triptolide (including its analogs). In addition to the CYP3A-mediated metabolization, triptolide is also identified as a substrate of P-glycoprotein. Knockdown of hepatic P-glycoprotein expression significantly altered the systemic and hepatic exposures of triptolide in vivo.

Accumulated clinical evidence discloses the functions and the toxicity of triptolide. Although triptolide is a promising drug candidate for clinics, its resource is greatly limited due to the trace amount of triptolide in the plant as well the tedious procedure of the extraction and purification from the plant. Thus, this disclosure provides a triptolide composition with increased bioavailability and reduced toxicity, especially hepatotoxicity. In some embodiments, the triptolide dosage in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of triptolide is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

Colchicine is a plant-based alkaloid that was originally extracted from Colchicum autumnale (autumn crocus, meadow saffron) and Gloriosa superba (glory lily). Colchicine has been approved to treat gout and some other inflammatory conditions such as familial Mediterranean fever and Behçet's Syndrome. A series of preclinical and clinical studies also showed that colchicine could prevent or improve cardiovascular disease. Chen et al., Am J Cardivasc Drugs, 17: 347-360 (2017). The therapeutic mechanism of colchicine's functions against diverse disorders is not fully understood, though it is known that the drug accumulates preferentially in leucocytes, particularly neutrophils, which is important for its therapeutic effect. Three major interactions of colchicine with specific proteins modulate its pharmacokinetics: tubulin, cytochrome P450 3A4 (CYP3A4), and P-glycoprotein. It is assumed that most therapeutic effects of the drug are related to its capacity to bind to beta-tubulin, thus inhibiting self-assembly and polymerization of microtubules. Availability of tubulin is essential for several cellular functions such as mitosis. Therefore, colchicine effectively functions as a “mitotic poison” or spindle poison. By inhibiting microtubule self-assembly, colchicine interferes with different cellular functions involved in the immune response such as modulation of the production of chemokines and prostanoids and inhibition of neutrophil and endothelial cell adhesion molecules. Eventually, it decreases neutrophil degranulation, chemotaxis, and phagocytosis, thus reducing the initiation and amplification of inflammation. Colchicine also inhibits uric acid crystal deposition (a process important to the genesis of gout), which is enhanced by a low pH in the tissues, probably by inhibiting oxidation of glucose and subsequent lactic acid reduction in leukocytes (Imazio, Brucato et al. 2009, Eur Heart J, 30(5): 532-9; Cocco, Chu et al. 2010, Eur J Intern Med, 21(6): 503-8; Stanton, Gernert et al. 2011, Med Res Rev, 31(3): 443-81). Colchicine suppresses the acute pericardial inflammation associated with pericarditis.

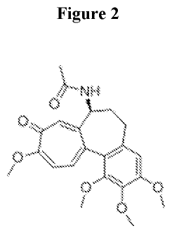

The chemical structure of colchicine and its analogs are shown in FIG. 2 and all such forms of colchicine and its pharmaceutically acceptable salts are contemplated herein, including hydrates, and co-crystals of such compounds and such salts. Examples of pharmaceutically acceptable salts include, but are not limited to, mineral or organic acid addition salts of basic residues such as amines, alkali or organic addition salts of acidic residues, and the like, and combinations comprising one or more of the foregoing salts. The pharmaceutically acceptable salts include non-toxic salts and the quaternary ammonium salts of the plant-based compounds. For example, non-toxic acid salts include those derived from inorganic acids such as hydrochloric, hydrobromic, sulfuric, sulfamic, phosphoric, nitric, and the like; other acceptable inorganic salts include metal salts such as sodium salt, potassium salt, cesium salt, and the like; alkaline earth metal salts, such as calcium salt, magnesium salt, and the like; and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH (where n is 0-4), and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparaginate, glutamate, and the like; and combinations comprising one or more of the foregoing salts. Specific colchicine salts include colchicine hydrochloride, colchicine dihydrochloride, and co-crystals, hydrates, or solvates thereof.

The pharmacokinetics of colchicine can be affected in several ways. The absorption of colchicine from the gastrointestinal tract is limited by the multidrug resistance efflux transporter P-glycoprotein. Like triptolide, colchicine is a substrate of intestinal and hepatic cytochrome CYP3A4, which catalyzes demethylation of colchicine to inactive metabolites. Without being bound by a theory, systemic concentrations of colchicine may be altered when it is co-administered with inhibitors of CYP3A4 and/or P-glycoprotein.

Colchicine's therapeutic values are limited by its narrow therapeutic index. The clinical features of colchicine toxicity appear in three phases, including gastrointestinal upset, and organ dysfunction. Therefore, the method to modulate pharmacokinetics and to increase the therapeutic index of colchicine can benefit patients suffering from a disease or condition that is targeted by colchicine. In some embodiments, the colchicine dosage pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of colchicine is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

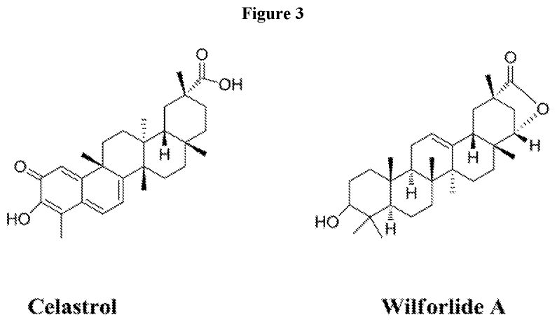

Other plant-based compounds that are applicable in this disclosure include, but are not limited to, glycosides (e.g., cardiac glycoside, cyanogenic glycoside, glucosinolate, saponin, and anthraquinone glycoside), wilforlide A, celastrol, flavonoids, proanthocyanidins, tannins, terpenoids (e.g. monoterpenoids, sesquiterpenoids, and phenylpropanoids), diterpenoids, resins, lignans, pyrrolizidine alkaloids, tropane alkaloids, alkaloids, furocoumarins, naphthodianthrones, and their derivatives and analogs. Among them, the structures of celastrol and wilforlide A are shown in FIG. 3.

Celastrol is a triterpene lactone epoxide compound, also known as a quinone-methide. Celastrol has been reported to inhibit growth and metastasis of melanoma, and to treat Alzheimer's disease. Wang et al., J Ethnopharmacol 194: 861-876 (2016).

The chemical structure of celastrol and its analogs are shown in FIG. 3 and all such forms of celastrol and its pharmaceutically acceptable salts are contemplated herein, including hydrates, and co-crystals of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the plant-based compounds. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH (where n is 0-4), and the like; organic amine salts such as triethylamine salt, pyridine salt, picoline salt, ethanolamine salt, triethanolamine salt, dicyclohexylamine salt, N,N′-dibenzylethylenediamine salt, and the like; amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; amino acid salts such as arginate, asparaginate, glutamate, and the like; and combinations comprising one or more of the foregoing salts. Specific celastrol salts include celastrol hydrochloride, celastrol dihydrochloride, and co-crystals, hydrates, or solvates thereof.

Like triptolide, celastrol is a substrate of intestinal and hepatic cytochrome CYP3A4. Without being bound by a theory, systemic concentrations of celastrol may be altered when it is co-administered with inhibitors of CYP3A4 and/or P-glycoprotein. In some embodiments, the celastrol dosage in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of celastrol in the pharmaceutical composition is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

Extracts from Schisandra sphenanthera

Schisandra sphenanthera, a native plant in China, has long been used as an ingredient in oriental medicine for treating viral and drug-induced hepatitis (Hancke J L et al., Fitoterapia, 70:451-471 (1999). The extracts from Schisandra sphenanthera, including gomisin A, can be hydroxylated by CYP3A, thereby serving as a potent substrate for this enzyme. Wu et al., AAPS. 1, 18(1), 134-45 (2016); CN 104892563A; Wu et al., Drug Metab Dispos, 42, 94-104 (2014). Further, the extracts from Schisandra sphenanthera can also increase the blood concentration of Tacrolimus (FK506), which is metabolized by CYP3A4, by inhibiting the enzymatic activity of CYP3A4. See Iwata et al., Drug Metab Dispos, 32, 1351-1358 (2004); Qin et al. Drug Metab Dispos, 32, 193-199 (2014).

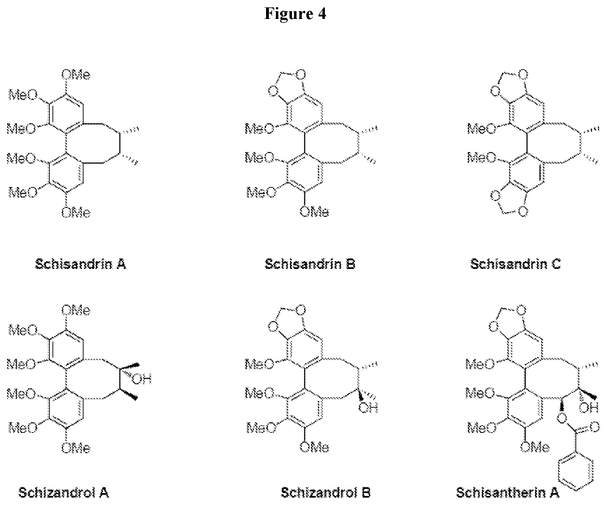

Without being bound by a theory, it is contemplated that the extracts of Schisandra sphenanthera can inhibit the activities of enzymes (e.g., CYP3A). In one embodiment, the extracts from Schisandra sphenanthera comprise a compound isolated from Schisandra sphenanthera. In another embodiment, the compound isolated from Schisandra sphenanthera includes Schisandrin A, Schisandrin B, Schisandrin C, Schizandrol A, Schizandrol B, Schisantherin A, or the combination thereof. The structures of the above compounds are shown in FIG. 4.

Drugs containing Schisandrin A as the major active ingredient have been approved in China for many years to protect liver function in patients with chronic hepatitis and liver dysfunction, e.g., Wuzhi Capsule (Sichuan Hezheng Pharmacy Co., Ltd. 11.25 mg of Schisandrin A/capsule, 2 capsules TID/day). Due to their proven bioactivities and safety, those drugs are widely used to treat viral and drug-induced hepatitis in China. Preclinical studies also demonstrated that compounds from Schisandra sphenanthera have a protective effect against cisplatin-induced nephrotoxicity by activating the Nrf2 mediated defense response, reducing the levels of reactive oxygen species (ROS), and increasing levels of glutathione (GSH). Recent study as well showed that compound from Schisandra sphenanthera can alleviate the symptoms of DSS-induced ulcerative colitis in mice via reducing the levels of inflammatory cytokines, suppressing CD4 T cell infiltration, and inhibiting the apoptosis in the colon.

All the pharmaceutically acceptable forms of Schisandrin A, Schisandrin B, Schisandrin C, Schizandrol A, Schizandrol B, Schisantherin A, and their pharmaceutically acceptable salts are contemplated herein, including hydrates, and co-crystals of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the plant-based compounds. For example, non-toxic acid salts include those derived from inorganic acids such as hydrochloric, hydrobromic, sulfuric, sulfamic, phosphoric, nitric, and the like; other acceptable inorganic salts include metal salts such as sodium salt, potassium salt, cesium salt, and the like; alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH (where n is 0-4), and the like; organic amine salts such as triethylamine salt, pyridine salt, picoline salt, ethanolamine salt, triethanolamine salt, dicyclohexylamine salt, N,N′-dibenzylethylenediamine salt, and the like; amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′ dibenzylethylenediamine salt, and the like; amino acid salts such as arginate, asparaginate, glutamate, and the like; and combinations comprising one or more of the foregoing salts.

In some embodiments, the dosage to achieve therapeutic effects of the Schisandra sphenanthera extract in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A is at least 0.1 mg/kg, at least 0.5 mg/kg, at least 1 mg/kg, at least 2 mg/kg, at least 3 mg/kg, at least 4 mg/kg, at least 5 mg/kg, at least 10 mg/kg, at least 15 mg/kg, at least 20 mg/kg, at least 30 mg/kg, 40 mg/kg, 50 mg/kg, at least 60 mg/kg, at least 70 mg/kg, at least 80 mg/kg, at least 90 mg/kg, or at least 100 mg/kg.

### Pharmaceutical Compositions

The disclosure provides that a combination of the compound originally extracted from Schisandra sphenanthera with a plant-based compound can increase the levels of the compound once administered. The compound originally extracted from Schisandra sphenanthera includes a compound that is synthesized but has the same or similar structure of the Schisandra sphenanthera extract. Surprisingly, applicants discovered that administration of Schisandrin A significantly increases systemic levels of the plant-based compound (e.g., triptolide, or colchicine, wilforlide A, celastrol, and their derivatives and analogs), and thus lowers the dosage of the plant-based compound that is normally used for medical purposes. Without being bound by a theory, the inhibition of CYP3A4/P-glycoprotein by a relatively large amount of Schisandrin A prevents or slows down the metabolism of triptolide or colchicine. Based on the noted percent change in Cₘₐₓ and AUC of triptolide or colchicine with Schisandrin A, the adjusted lower doses of plant compounds (e.g., triptolide or colchicine) bring about the similar treating function but with less toxicity comparing to the original dose. Furthermore, the disclosure provides that the combination of Schisandrin A with plant compounds (e.g., triptolide or colchicine) could significant attenuate the systemic toxicity caused by triptolide or colchicine.

Schisandrin A and its analogs from Schisandra sphenanthera are potent substrates of CYP3A and thus can act as a competing inhibitor of CYP3A4. Furthermore, compounds from Schisandra sphenanthera can act as P-glycoprotein inhibitors and restore the cytotoxic effects of doxorubicin to cancer cell lines. Thus, this disclosure provides compositions that enhance the clinical utility of plant-based compounds, including colchicine and triptolide.

In one aspect, the disclosure provides a pharmaceutical composition, wherein the pharmaceutical composition comprises, alternatively consists essentially of, or yet consists of an extract from Schisandra sphenanthera and a plant-based compound. In one embodiment, the plant-base compound comprises one or more of triptolide, colchicine, and their derivatives and analogs. Non-limiting examples of triptolide analogs include 16-hydroxy-triptolide, triptonide, and tripdiolide. In one embodiment, the plant-based compounds comprise one or more of glycosides (e.g., cardiac glycoside, cyanogenic glycoside, glucosinolate, saponin, and anthraquinone glycoside), wilforlide A, celastrol, flavonoids, proanthocyanidins, tannins, terpenoids (e.g., monoterpenoids, sesquiterpenoids, and phenylpropanoids), diterpenoids, resins, lignans, pyrrolizidine alkaloids, tropane alkaloids, alkaloids, furocoumarins, naphthodianthrones, and their derivatives and analogs. In a different embodiment, the plant-based compound comprises one or more of wilforlide A, celastrol, and their derivatives and analogs. In another embodiment, the extract from Schisandra sphenanthera comprises one or more of Schisandrin A, Schisandrin B, Schisandrin C, Schizandrol A, Schizandrol B, and Schisantherin A.

The dosages of a plant-based compound (e.g., triptolide or colchicine) can vary among patients due to their low dosages and being a substrate for both CYP3A4 and the P-glycoprotein. In some embodiments, the dosage of plant-based compounds is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of plant-based compounds is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In some embodiments, the dosage of triptolide and its derivatives or analogs compounds is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of triptolide and its derivatives or analogs compounds is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg. In one aspect, triptolide analogs comprise one or more of 16-hydroxy-triptolide, triptonide, and tripdiolide.

In some embodiments, the colchicine dosage pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of colchicine is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In some embodiments, the celastrol dosage in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of celastrol in the pharmaceutical composition is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In one aspect, the pharmaceutical composition further comprises, alternatively consists essentially of, or yet consists of an inhibitor of a CYP enzyme. Non-limiting examples of CYP enzymes include CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, and CYP4Z1. Thus, in one embodiment, the CYP inhibitor is a CYP3A inhibitor. Non-limiting examples of substrates or inhibitors for CYP enzyme can be found at http://www.genemedrx.com/Cytochrome_P450_Metabolism_Table.php. Non-limiting examples of CYP enzyme inhibitor includes amiodarone, amprenavir, aprepitant, REYATAZ® (atazanavir), cimetidine, ciprofloxacin, clarithromycin, delavirdine, diltiazem, doxycycline, echinacea, enoxacin, erythromycin, fluconazole, fluvoxamine, grapefruit juice, indinavir, itraconazole, ketoconazole, miconazole, nefazodone, nelfinavir, ritonavir, saquinavir, telithromycin, verapamil, and voriconazole. A CYP3A is one of the CYP enzymes, which is found in the liver and the intestine.

In some embodiments, the CYP3A inhibitor comprises, alternatively consists essentially of, or yet consists of one or more of ketoconazole, itraconazole, fluconazole, cimetidine, clarithromycin, erythromycin, troleandomycin, and grapefruit juice.

In some embodiments, the pharmaceutical composition comprises a p-glycoprotein inhibitor. Non-limiting examples of p-glycoprotein inhibitors include amiodarone, clarithromycin, erythromycin, ketoconazole, quinidine, saquinavir, and verapamil.

In one embodiment, the dosage of the extract from Schisandra sphenanthera in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A is at least 0.1 mg/kg, at least 0.5 mg/kg, at least 1 mg/kg, at least 2 mg/kg, at least 3 mg/kg, at least 4 mg/kg, at least 5 mg/kg, at least 10 mg/kg, at least 15 mg/kg, at least 20 mg/kg, at least 30 mg/kg, 40 mg/kg, 50 mg/kg, at least 60 mg/kg, at least 70 mg/kg, at least 80 mg/kg, at least 90 mg/kg, or at least 100 mg/kg. In one embodiment, the mass ratio of the Schisandra sphenanthera extract to the plant-based compound is at least 3:1, 6:1, 12:1, 24:1, or 30:1.

A pharmaceutical composition is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

In one embodiment, compositions can be formulated orally in the form of tablets, capsules, cachets, gelcaps, solutions, or suspensions. The tablets may be coated by methods well-known in the art. Liquid preparations for oral administration include, but are not limited to, solutions, syrups, or suspensions, or they may be presented as a dry product for constitution with water or other suitable vehicle before use.

In some embodiments, the composition for oral administration further comprises one or more of binding agents, flavor agents, lubricating agents, flow agents, disintegration agents, delay agents, and organic solvents. In some embodiments, the binding agents for the oral composition comprise starch, modified starch, cellulose, modified cellulose, brewer's yeast, sucrose, dextrose, whey, and dicalcium phosphate. In some embodiments, the lubricating agents comprise magnesium stearate, stearic acid, starch, modified starch, and modified cellulose. In some embodiments, of the oral composition the flow agents comprise silica dioxide, modified silica, fumed silica, and talc. In some embodiments, the disintegration agents comprise croscarmellose sodium, sodium starch glycolate, starch, and modified starch. In some embodiments, the delay agents comprise one or more of stearic acid, stearic acid salts, magnesium stearate, polyethylene glycols, starch, modified starch, and methacrylate polymers. In some embodiments, the organic solvents comprise propylene glycol, polyethylene glycols, ethanol, dimethyl sulfoxide (DMSO), N-methyl-2-pyrrolidone, glycofurol, Solketal, glycerol formal, acetone, tetrahydrofurfuryl alcohol, diglyme, dimethyl isosorbide, and ethyl lactate. In some embodiments, the concentration of the organic solvent is 0.1% to about 35% of the total volume of the composition. In some embodiments, the concentration of the organic solvent is 2% of the total volume of the composition.

In another embodiment, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., (Mack Publishing Co., Easton, Pa., 1995). For non-sprayable topical dosage forms, viscous to semi-solid or solid forms comprising a carrier or one or more excipients compatible with topical application and having a dynamic viscosity preferably greater than water are typically employed. Other suitable formulations include, without limitation, suspensions, powders, liniments, salves, and the like. In one embodiment, such formulations are sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, for example, in combination with a solid or liquid inert carrier, is packaged in a mixture with a pressurized volatile (e.g., a gaseous propellant, such as FREON®) or in a squeeze bottle. Moisturizers or humectants can also be added to pharmaceutical compositions and dosage forms if desired. Examples of such additional ingredients are well-known in the art. In some embodiments, the formulation for topical administration further comprises organic solvents. In some embodiments, the organic solvent comprises propylene glycol, polyethylene glycols, ethanol, DMSO, N-methyl-2-pyrrolidone, glycofurol, Solketal, glycerol formal, acetone, tetrahydrofurfuryl alcohol, diglyme, dimethyl isosorbide, and ethyl lactate. In some embodiments, the concentration of the organic solvent is 0.1% to about 35% of the total volume of the composition. In some embodiments, the concentration of the organic solvent is 2% of the total volume of the composition.

In one embodiment, the composition can be formulated in aerosol, spray, mist, or in the form of drops. In particular, prophylactic or therapeutic agents can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch. In some embodiments, the pharmaceutical composition for intranasal administration further comprises one or more of organic solvents, suspending agents, isotonicity agents, buffers, emulsifiers, stabilizers, and preservatives. In some embodiments, the organic solvent of the intranasal composition comprises one or more of propylene glycol, polyethylene glycols, ethanol, DMSO, N-methyl-2-pyrrolidone, glycofurol, Solketal, glycerol formal, acetone, tetrahydrofurfuryl alcohol, diglyme, dimethyl isosorbide, and ethyl lactate. In some embodiments, the concentration of the organic solvent is 0.1% to about 35% of the total volume of the composition. In some embodiments of the intranasal composition, the concentration of the organic solvent is 2% of the total volume of the composition. In some embodiments of the intranasal composition, the suspending agents comprise one or more of carbomer, carboxymethyl cellulose sodium, poloxamers, povidone, microcrystalline cellulose, polyvinyl alcohol, methylhydroxy ethyl cellulose, hydroxypropyl methyl cellulose, hydroxyethylcellulose, hydroxypropylcellulose, polycarbophils, xanthan gum, and guar gum. In some embodiments of the intranasal composition, the isotonicity agents comprise one or more of sodium chloride, mannitol, and glycerol. In some embodiments of the intranasal composition, the buffers comprise one or more of phosphate-citrate buffer, phosphate buffer, citrate buffer, histidine acetate, histidine-histidine hydrochloride, L-Histidine, L-Argenine hydrochloride, bicarbonate buffer, succinate buffer, citrate buffer, and TRIS buffer. In some embodiments of the intranasal composition, the emulsifiers comprise one or more of polyoxyl-35-castor oil, glycerine stearate and polyethyleneglycol 75 stearate, polyoxyl-40-hydrogenated castor oil, polyethylene glycol-6-32-stearate and glycol stearate, sorbitan trioleate, oleic acid, phospholipids such as phosphatidylethanolamine, phosphatidylchloline, and phosphatidylinositol. In some embodiments of the intranasal composition, the emulsifier is present in a concentration ranging from 0.001 to about 30%. In some embodiments of the intranasal composition, the stabilizers comprise one or more of hydroxypropyl beta cyclodextrin, gamma cyclodextrin, sodium metabisulphite, sodium sulphite, sodium bisulphite, acetyl cysteine, butylated hydroxyanisole, butylated hydroxytoluene, propyl gallate, tocopheryl compounds, and d-alpha tocopheryl polyethylene glycol 1000 succinate. In some embodiments of the intranasal composition, the preservatives comprise one or more of potassium sorbate, benzalkonium chloride, phenylethylalcohol, methylparaben, propylparaben, ethylparaben, butylparaben, disodium edetate, sorbic acid, and phenoxyethanol.

The composition may be formulated as a sterile aqueous solution suitable for injection intravenously, subcutaneously, intraperitoneally, or intramuscularly.

Bioavailability is a measure of the relative amount of a drug administered in a pharmaceutical product that enters the systemic circulation in an unchanged form, and the rate at which this occurs. See Principles of Clinical Pharmacology edited by Atkinson et al. (Academic Press, 2001). Therefore, bioavailability of drugs depends not only on the absorption rate and elimination rate of the drug, but also on how the drug interacts with and is changed by metabolic enzymes, transmembrane transporter proteins, and other molecules involved in the metabolism and transport of molecules in animal and human physiological system.

The compounds disclosed herein can be administered in combination or alternation with a second biologically active agent to increase its effectiveness against the target disorder. In combination therapy, effective dosages of two or more agents are administered together, whereas during alternation therapy an effective dosage of each agent is administered serially. The dosages will depend on absorption, inactivation and elimination rates of the drug as well as other factors known to those with ordinary skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The efficacy of a drug can be prolonged, augmented, or restored by administering the compound in combination or alternation with a second, and perhaps third, agent that induces a different biological pathway from that caused by the principle drug. Alternatively, the pharmacokinetics, biodistribution, or other parameter of the drug can be altered by such combination or alternation therapy. In general, combination therapy is typically preferred over alternation therapy because it induces multiple simultaneous stresses on the condition.

In some embodiments, the extract of Schisandra sphenanthera is administered for a period of time sufficient to reduce or attenuate the activity of the enzyme against plant-based compounds such that the extract of Schisandra sphenanthera has an anti-enzymatic activity thereby increasing the bioavailability of plant-based compounds in the subject. In some embodiments, the dosage of the extract from Schisandra sphenanthera in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A is at least 0.1 mg/kg, at least 0.5 mg/kg, at least 1 mg/kg, at least 2 mg/kg, at least 3 mg/kg, at least 4 mg/kg, at least 5 mg/kg, at least 10 mg/kg, at least 15 mg/kg, at least 20 mg/kg, at least 30 mg/kg, 40 mg/kg, 50 mg/kg, at least 60 mg/kg, at least 70 mg/kg, at least 80 mg/kg, at least 90 mg/kg, or at least 100 mg/kg. In one embodiment, the mass ratio of the Schisandra sphenanthera extract to the plant-based compound is at least 3:1, 6:1, 12:1, 24:1, or 30:1.

In some embodiments, the Schisandra sphenanthera extract is administered to increase the bioavailability of the plant-based compound triptolide. In some embodiments, the dosage of triptolide and its derivatives or analogs compounds is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of triptolide and its derivatives or analogs compounds is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg. In one aspect, triptolide analogs comprise one or more of 16-hydroxy-triptolide, triptonide, and tripdiolide.

In some embodiments, the Schisandra sphenanthera extract is administered to increase the bioavailability of the plant-based compound colchicine. In some embodiments, the colchicine dosage pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of colchicine is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In some embodiments, the Schisandra sphenanthera extract is administered to increase the bioavailability of the plant-based compound celastrol. In some embodiments, the celastrol dosage in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of celastrol in the pharmaceutical composition is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In some embodiments, the plant-based compound and the Schisandra sphenanthera extract are administered separately, simultaneously, or sequentially.

Many metabolic reactions that change the form of drugs involve cytochrome P (CYP) enzymes. In particular, CYP1, CYP2, and CYP3 families are thought to be important in the metabolism of drugs and CYP3A4 is the most abundant member of these CYP families. Without being bound by theory, it is believed that Schisandra sphenanthera can increase bioavailability of drugs by inhibiting enzymes that metabolize drugs such as CYP family enzymes. Hence, in some embodiments, the pharmaceutical composition comprises Schisandra sphenanthera extract.

In some embodiments, the bioavailability of a plant-based compound (e.g., triptolide, colchicine, wilforlide A, celastrol, and their derivatives and analogs) is increased due to administration of other Cytochrome P (CYP) and P-glycoprotein (P-gp) inhibitors. Thus, the method comprises administering an inhibitor of a CYP enzyme. In some embodiments, the CYP enzyme inhibitor is a CYP3A inhibitor. Non-limiting examples of CYP enzymes include CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, and CYP4Z1. Thus, in one embodiment, the CYP inhibitor is a CYP3A inhibitor. Non-limiting examples of substrates or inhibitors for CYP enzyme can be found at http://www.genemedrx.com/Cytochrome_P450_Metabolism_Table.php. Non-limiting examples of CYP enzyme inhibitors includes amiodarone, amprenavir, aprepitant, REYATAZ® (atazanavir), cimetidine, ciprofloxacin, clarithromycin, delavirdine, diltiazem, doxycycline, echinacea, enoxacin, erythromycin, fluconazole, fluvoxamine, grapefruit juice, indinavir, itraconazole, ketoconazole, miconazole, nefazodone, nelfinavir, ritonavir, saquinavir, telithromycin, verapamil, and voriconazole. A CYP3A is one of the CYP enzymes, which is found in the liver and the intestine. Non-limiting examples of CYP3A inhibitors include ketoconazole, itraconazole, fluconazole, cimetidine, clarithromycin, erythromycin, troleandomycin, and grapefruit juice. In some embodiments, the CYP3A inhibitor comprises, alternatively consists essentially of, or yet consists of one or more of ketoconazole, itraconazole, fluconazole, cimetidine, clarithromycin, erythromycin, troleandomycin, and grapefruit juice. Non-limiting examples of p-glycoprotein inhibitor include amiodarone, clarithromycin, erythromycin, ketoconazole, quinidine, saquinavir, and verapamil.

The bioavailability of a plant-based compound (e.g., triptolide, colchicine, wilforlide A, celastrol, and their derivatives and analogs) is increased by administration of inhibitors of P-glycoprotein (P-gp). Transporter proteins in the cellular membranes are important for the absorption, distribution, and elimination of many drugs, and can therefore decrease bioavailability of drugs. For example P-glycoprotein restricts drug entry into and through the intestinal lumen, thereby decreasing drug availability. Thus, in some embodiments, the method comprises administering an inhibitor of a P-glycoprotein.

Without being bound be theory, it is believed that the methods and compositions disclosed herein will increase the bioavailability of the plant-based compound due to the pharmaceutical composition described above by inhibiting CYP family enzymes and/or the P-glycoprotein transporter. In some embodiments, the bioavailability of the plant-based compound due to administration of the pharmaceutical composition is increased by at least 10%, 30%, 60%, or 100% compared to the plant-based compound without administration of the pharmaceutical composition. In some embodiments, the bioavailability of the plant-based compound due to administration of the pharmaceutical composition is increased by at least 50% compared to the plant-based compound without administration of the pharmaceutical composition.

### Methods of Treatment

In one aspect, the disclosure provides methods of treating and/or preventing a disease in a subject, comprising, alternatively consisting essentially of, or yet consisting of administering to the subject an effective amount of a pharmaceutical composition, said pharmaceutical composition comprises an extract from Schisandra sphenanthera, and a plant-based compound. In some embodiments, the subject is a human patient. In some embodiments, the subject is a mammal. In some embodiments, the subject is a cat, a dog, a rabbit, a cow, or a pig. In some embodiments, the disease is selected from a group consisting of autoimmune diseases, neurodegenerative disorders (e.g. Alzheimer's disease), transplantation rejection, cancers (e.g. pancreatic cancer, renal cancer, small cell lung cancer, brain cancer, neural cancer, bone cancer, lymphoma, colon cancer, uterine cancer, breast cancer, leukemia, liver cancer, prostate cancer, skin cancer, and melanoma), infertility, gout, familial Mediterranean fever, cardiovascular diseases, Behçet's disease, and anti-inflammatory disorders or the symptoms thereof.

In some embodiments, the plant-based compound comprises, alternatively consists essentially of, or yet consists of one or more of triptolide, colchicine, and their derivatives or analogs. In some embodiments, the extract from Schisandra sphenanthera comprises, alternatively consists essentially of, or yet consists of one or more of Schisandrin A, Schisandrin B, Schisandrin C, Schizandrol A, Schizandrol B, and Schisantherin A. In some embodiments, the dosage of the extract from Schisandra sphenanthera in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments, the dosage of Schisandrin A in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg. In some embodiments the dosage of Schisandrin A is at least 0.1 mg/kg, at least 0.5 mg/kg, at least 1 mg/kg, at least 2 mg/kg, at least 3 mg/kg, at least 4 mg/kg, at least 5 mg/kg, at least 10 mg/kg, at least 15 mg/kg, at least 20 mg/kg, at least 30 mg/kg, 40 mg/kg, 50 mg/kg, at least 60 mg/kg, at least 70 mg/kg, at least 80 mg/kg, at least 90 mg/kg, or at least 100 mg/kg. In one embodiment, the mass ratio of the Schisandra sphenanthera extract to the plant-based compound is at least 3:1, 6:1, 12:1, 24:1, or 30:1.

In one embodiment, the plant-based compound (e.g., triptolide, colchicine, wilforlide A, celastrol, and their derivatives and analogs) and the extract from Schisandra sphenanthera are administered separately, simultaneously, or sequentially. In some embodiments, the plant-based compound (e.g., triptolide, colchicine, wilforlide A, celastrol, and their derivatives and analogs) is administered before, during, and/or after administration of the extract from Schisandra sphenanthera. In some embodiments, the plant-based compound (e.g., triptolide, colchicine, wilforlide A, celastrol, and their derivatives and analogs) is administered between one minute and 24 hours prior to administration of the extract of Schisandra sphenanthera. In some embodiments, the plant-based compound is administered 1, 2, 3, 4, 5, 6, or 7 days prior to administration of the extract of Schisandra sphenanthera.

The described derivative of triptolide (including its derivatives and analogs) can be formulated as pharmaceutical compositions and administered for any of the disorders described herein, and in particular for treating cancer in a subject. In some embodiments triptolide and its derivatives are administered for treating pancreatic cancer, renal cancer, small cell lung cancer, brain cancer, neural cancer, bone cancer, lymphoma, colon cancer, uterine cancer, breast cancer, leukemia, liver cancer, prostate cancer, skin cancer, and melanoma in a subject. In some embodiments, the dosage of triptolide and its derivatives or analogs compounds is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of triptolide and its derivatives or analogs compounds is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg. In one aspect, triptolide analogs comprise one or more of 16-hydroxy-triptolide, triptonide, and tripdiolide.

The described plant-based compound colchicine can be formulated as pharmaceutical compositions and administered for any of the disorders described herein and, in particular, for treating cardiovascular disease in a subject. In some embodiments, the colchicine dosage pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of colchicine is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

The described plant-based compound celastrol can be formulated as pharmaceutical compositions and administered for any of the disorders described herein and, in particular, for treating neurodegenerative disease or cancer in a subject. In some embodiments, celastrol is administered for treating Alzheimer's disease. In some embodiments, celastrol is administered for treating melanoma. In some embodiments, the celastrol dosage in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of celastrol in the pharmaceutical composition is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In one embodiment, the extract of Schisandra sphenanthera and plant-based compounds (e.g., triptolide, colchicine, wilforlide A, celastrol, and their derivatives and analogs) are administered intravenously, subcutaneously, orally, or intraperitoneally. In a preferred embodiment, the extract of Schisandra sphenanthera is administered proximal to (e.g., near or within the same body cavity as) the organ(s) and/or tissue(s) infected by the diseases. In one embodiment, the extract is administered directly into a blood vessel feeding the infected organ(s) and/or tissue(s). In one embodiment, the extract is administered systemically. In a further embodiment, the extract is administered by microcatheter, an implanted device, or an implanted dosage form.

In one embodiment, the extract of Schisandra sphenanthera is administered in a continuous manner for a defined period. In another embodiment, the extract of Schisandra sphenanthera is administered in a pulsatile manner. For example, the extract of Schisandra sphenanthera may be administered intermittently over a period of time.

In another embodiment, the extract from Schisandra sphenanthera is administered prior to administration of the plant-based compound (e.g., triptolide, colchicine, wilforlide A, celastrol, and their derivatives and analogs). In one embodiment, the extract from Schisandra sphenanthera is administered after administration of the plant-based compound. In one embodiment, the extract from Schisandra sphenanthera is administered before, during, and/or after administration of the plant-based compound.

During the treatment, the pharmaceutical composition is included in the pharmaceutically acceptable carrier or diluent in an amount sufficient to deliver to a patient a therapeutically effective amount of compound to treat autoimmune diseases, neurodegenerative disorders (e.g. Alzheimer's disease), transplantation rejection, cancers (e.g. pancreatic cancer, renal cancer, small cell lung cancer, brain cancer, neural cancer, bone cancer, lymphoma, colon cancer, uterine cancer, breast cancer, leukemia, liver cancer, prostate cancer, skin cancer, and melanoma), infertility, gout, familial Mediterranean fever, cardiovascular diseases, Behçet's disease, and anti-inflammatory disorders or the symptoms thereof in vivo without causing serious toxic effects in the patient treated.

As noted above, the concentration of the plant-based compound (e.g., triptolide, colchicine, wilforlide A, celastrol, and their derivatives and analogs) in the pharmaceutical composition will depend on absorption, inactivation, and excretion rates of the extract as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The plant-based compound (e.g., triptolide, colchicine, wilforlide A, celastrol, and their derivatives and analogs) may be administered at once, or may be divided into a number of smaller doses to be administered at varying intervals of time.

### Dose and Administration

The pharmaceutical compositions, as described herein, are administered in effective amounts for treating the herein disclosed diseases. The effective amount will depend upon the mode of administration, the particular condition being treated, and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result. A person of ordinary skill in the art will appreciate that dosages determined by animal experiments can be converted an equivalent dosage for a different animal species or human. See, e.g. Nair et al., J. Basic Clin. Pharm. 7: 27-31 (2016). For example, a dosage for an animal species can be converted an equivalent dosage for human based on the conversion table in Nair et al., J. Basic Clin. Pharm. 7: 27-31 (2016).

Generally, the dose of the plant-based compound (e.g., triptolide, colchicine, wilforlide A, celastrol, and their derivatives and analogs) is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg. In some embodiments, the dosage of the plant-based compound and its derivatives or analogs compounds is at least 0.01 mg/kg, at least 0.02 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.27 mg/kg, at least 0.8 mg/kg, at least 0.9 mg/kg, at least 1 mg/kg, at least 2 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, or at least 100 mg/kg.

In one aspect of the invention, administration of the pharmaceutical composition as described herein is pulsatile. In one embodiment, an amount of pharmaceutical composition is administered every 1 hour to every 24 hours, for example, every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In one embodiment, an amount of pharmaceutical composition is administered every 1 day, 2 days, 3 days, 4 days, 5 days, 6 days, 7 days, 8 days, 9 days, or 10 days.

A variety of administration routes are available. The pharmaceutical composition of the invention, generally speaking, may be practiced using any mode of administration that is medically acceptable, meaning any mode that produces effective levels of the active ingredients without causing clinically unacceptable adverse effects.

Modes of administration include oral, rectal, topical, nasal, intradermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

Compositions suitable for oral administration may be presented as discrete units, such as capsules, tablets, or lozenges, each containing a predetermined amount of the active agent(s). Other compositions include suspensions in aqueous liquids or non-aqueous liquids such as a syrup, an elixir, or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Other delivery systems can include time-release, delayed-release, or sustained-release delivery systems. Such systems can avoid repeated administrations of the pharmaceutical composition of this invention, increasing convenience to the subject and the physician. Many types of release delivery systems are available and known to those of ordinary skill in the art. They include polymer-based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids, including sterols such as cholesterol, cholesterol esters, and fatty acids or neutral fats such as mono-, di-, and tri-glycerides; hydrogel release systems; sylastic systems; peptide-based systems; wax coatings; compressed tablets using conventional binders and excipients; partially fused implants; and the like.

In one embodiment, the pharmaceutical composition is administered in a time-release, delayed-release, or sustained-release delivery system. In one embodiment, the time-release, delayed-release, or sustained-release delivery system comprising the pharmaceutical composition of the invention is inserted directly into the tumor.

When administered, the pharmaceutical preparations of the invention are applied in pharmaceutically acceptable amounts and in pharmaceutically acceptable compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium, or calcium salts.

### Kit of Parts

In one aspect, this invention relates to a kit of parts for treatment of a disease in a subject, the kit comprising an extract from Schisandra sphenanthera, and a plant-based compound. In one embodiment, the disease is selected from a group consisting of autoimmune diseases, transplantation rejection, cancers, infertility, gout, familial Mediterranean fever, cardiovascular diseases, and Behçet's disease.

In another embodiment, the plant-base compound comprises, alternatively consists essentially of, or yet consists of one or more of triptolide, colchicine, wilforlide A, celastrol, and their derivatives and analogs. In some embodiments, the extract from Schisandra sphenanthera comprises, alternatively consists essentially of, or yet consists of one or more of Schisandrin A, Schisandrin B, Schisandrin C, Schizandrol A, Schizandrol B, and Schisantherin A.

In some embodiments, the kit further comprises, alternatively consists essentially of, or yet consists of an inhibitor of a CYP enzyme and/or a p-glycoprotein inhibitor.

In one embodiment, the kit further comprises instructions for treating the disease. In one embodiment, the kit of parts comprises instructions for dosing and/or administration of the pharmaceutic composition of this invention.

### WORKING EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

Animal Treatments:

42 Male Sprague Dawley rats (weight, 220-280 g) were maintained on a 12-hour light/dark cycle with free access to water and lab chow for 10-14 hours prior to the experiment. Rats were randomly divided into 7 groups (n=6/group) and groups 2-7 received 0 mg/kg, 6.0 mg/kg, and 12.0 mg/kg, 24.0 mg/kg, 48.0 mg/kg and 60.0 mg/kg of Schisandrin A in 1% CMC-Na (300-800 cps) via oral administration, respectively. After 5 minutes, all the groups received 2.4 mg/kg of triptolide in a solution of 2% DMSO-98% Sterile Water via oral administration.

Kinetic Study:

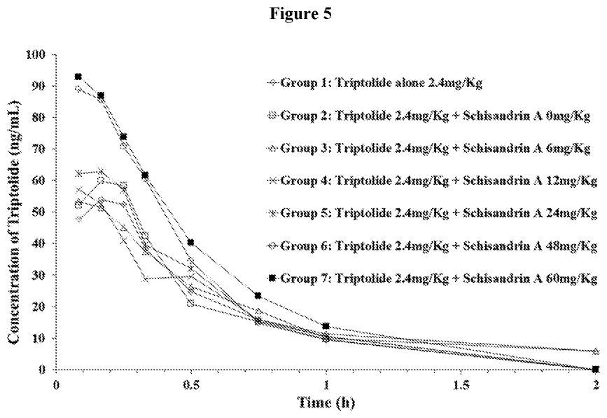

After triptolide treatment, blood samples were collected from individual rat at 5 min, 10 min, 15 min, 20 min, 30 min, 45 min, 1 h, 2 h, 4 h, 8 h, and 24 h, respectively. Plasma was separated by centrifugation at 8000 rpm for 6 minutes at 4° C. and kept at −80° C. until analysis. Plasma homogenate was injected into LC-MS/MS for analysis. Plasma triptolide concentration-time profiles in different group rats are shown in FIG. 5. Pharmacokinetic parameters of triptolide after a single oral dose of triptolide (2.4 mg/kg) in rats with and without different dose of Schisandrin A, including area under the concentration-time curve (AUC), mean residence time (MRT) and terminal elimination half-life (T_{1/2}), Cₘₐₓ, and Tₘₐₓ are as shown in Table 1.

TABLE 1
Pharmacokinetic parameters of triptolide after a single oral dose of triptolide (2.4 mg/kg)
in rats with and without different dose of Schisandrin A. Data are the mean ± S.D. (n = 6).

| | Pharmacokinetic parameters of triptolide | | | | | | |
|---|---|---|---|---|---|---|---|
| | T_{1/2} | Tₘₐₓ | Cₘₐₓ | AUC_{0→t} | AUC_{0→∞} | MRT_{0→t} | MRT_{0→∞} |
| Group | hr | hr | ng/mL | hr*ng/mL | hr*ng/mL | hr | hr |
| Group1 | 0.5 ± 0.19 | 0.181 ± 0.063 | 55.51 ± 18.22 | 30.47 ± 6.35 | 30.47 ± 6.35 | 0.45 ± 0.15 | 0.73 ± 0.24 |
| Group2 | 0.38 ± 0.15 | 0.195 ± 0.068 | 62.85 ± 15.20 | 28.33 ± 5.56 | 28.33 ± 5.56 | 0.35 ± 0.03 | 0.56 ± 0.17 |
| Group3 | 0.42 ± 0.23 | 0.166 ± 0.090 | 56.43 ± 25.13 | 31.53 ± 25.44 | 31.53 ± 25.44 | 0.43 ± 0.16 | 0.66 ± 0.35 |
| Group4 | 0.26 ± 0.06 | 0.167 ± 0.167 | 63.72 ± 29.15 | 26.81 ± 16.13 | 26.81 ± 16.13 | 0.34 ± 0.08 | 0.42 ± 0.09 |
| Group5 | 0.29 ± 0.04 | 0.153 ± 0.082 | 64.61 ± 24.59 | 31.57 ± 11.68 | 35.87 ± 12.49 | 0.35 ± 0.03 | 0.49 ± 0.06 |
| Group6 | 0.26 ± 0.05 | 0.125 ± 0.046 | 91.72 ± 39.43 | 40.04 ± 13.08 | 43.69 ± 12.76 | 0.34 ± 0.03 | 0.43 ± 0.07 |
| Group7 | 0.29 ± 0.07 | 0.111 ± 0.043 | 94.76 ± 47.33 | 47.93 ± 24.32 | 51.78 ± 24.16 | 0.39 ± 0.11 | 0.49 ± 0.13 |

Toxicological Study:

At 24 hours of triptolide treatment, blood was collected from the rats in plasma kinetic study. Serum ALT, AST, creatinine, and urea levels were determined as shown in Table 2.

TABLE 2
Serum chemistry parameters) in rats after a single oral dose of triptolide (2.4 mg/kg
with and without different dose of Schisandrin A. Data are the mean ± S.D. (n = 6).

| | Group 1 | Group 2 | Group 3 | Group 4 | Group 5 | Group 6 |
|---|---|---|---|---|---|---|
| ALT | 160.12 ± 88.64 | 122.48 ± 28.82 | 94.10 ± 19.81 | 83.26 ± 22.75 | 221.76 ± 183.22 | 155.96 ± 88.67 |
| (U/L) | | | | | | |
| AST | 508.60 ± 322.98 | 398.00 ± 270.93 | 304.80 ± 142.42 | 266.60 ± 154.80 | 666.20 ± 657.30 | 517.20 ± 354.59 |
| (U/L) | | | | | | |
| Creatinine | 0.89 ± 0.81 | 0.54 ± 0.61 | 0.49 ± 0.02 | 0.51 ± 0.0.02 | 0.792 ± 0.590 | 0.85 ± 0.58 |
| (mg/dL) | | | | | | |
| Urea | 78.06 ± 100.41 | 41.17 ± 20.06 | 27.76 ± 5.77 | 31.48 ± 9.20 | 79.88 ± 113.68 | 103.72 ± 105.01 |
| (mg/dL) | | | | | | |

### Example 2

Animal Treatments:

42 Male Sprague Dawley rats (weight, 210-250 g) were maintained on a 12-hour light/dark cycle with free access to water and lab chow for 10-14 h prior to the experiment. Rats were randomly divided into 7 groups (n=6 each) and groups 2-7 received 0 mg/kg, 5.5 mg/kg, and 11.0 mg/kg, 22.0 mg/kg, 44.0 mg/kg and 88.0 mg/kg of Schisandrin A in 1% CMC-Na (300-800 cps) via oral administration, respectively. After 5 minutes, all the groups received 1.8 mg/kg of celastrol in a solution of 2% DMSO-98% sterile water via oral administration.

Kinetic Study:

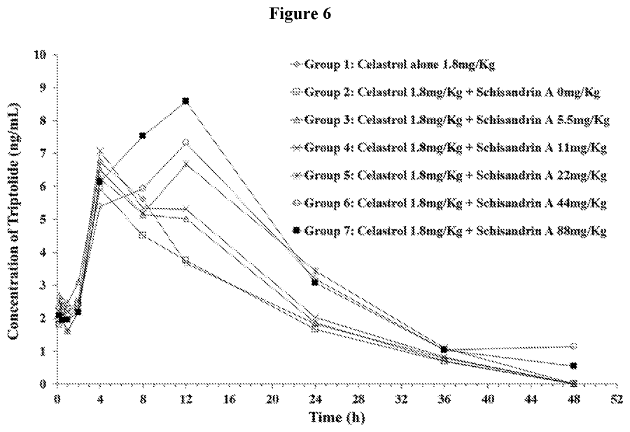

After celastrol treatment, blood samples were collected from individual rat at 0.25 h, 0.5 h, 1 h, 2 h, 4 h, 8 h, 12 h, 24 h, 36 h, and 48 h, respectively. Plasma was separated by centrifugation at 8000 rpm for 6 minutes at 4° C. and kept at −80° C. until analysis. Plasma homogenate was injected into LC-MS/MS for analysis. Plasma celastrol concentration-time profiles in different groups are shown in FIG. 6. Pharmacokinetic parameters of celastrol after a single oral dose of celastrol (1.8 mg/kg) in rats with and without different dose of Schisandrin A, including area under the concentration-time curve (AUC), mean residence time (MRT) and terminal elimination half-life (T_{1/2}), Cₘₐₓ, and Tₘₐₓ are shown in Table 3.

TABLE 3
Pharmacokinetic parameters of celastrol after a single oral dose of celastrol (1.8 mg/kg)
in rats with and without different dose of Schisandrin A. Data are the mean ± S.D. (n = 6).

| | Pharmacokinetic parameters of Celastrol | | | | | | |
|---|---|---|---|---|---|---|---|
| | T_{1/2} | Tₘₐₓ | Cₘₐₓ | AUC_{0→t} | AUC_{0→∞} | MRT_{0→t} | MRT_{0→∞} |
| Group | hr | hr | ng/mL | hr*ng/mL | hr*ng/mL | hr | hr |
| Group1 | 10.07 ± 2.48 | 5.33 ± 2.07 | 7.03 ± 2.65 | 102.66 ± 28.23 | 124.56 ± 22.44 | 12.06 ± 1.74 | 12.06 ± 3.49 |
| Group2 | 12.06 ± 1.77 | 4.00 ± 0.00 | 5.94 ± 1.38 | 93.21 ± 2.74 | 106.80 ± 19.64 | 12.00 ± 1.82 | 12.00 ± 2.33 |
| Group3 | 11.47 ± 5.18 | 5.60 ± 3.58 | 7.13 ± 1.86 | 117.02 ± 27.81 | 124.29 ± 27.89 | 13.12 ± 2.27 | 18.80 ± 8.57 |
| Group4 | 9.10 ± 1.71 | 4.00 ± 0.00 | 7.08 ± 0.57 | 123.01 ± 20.06 | 132.82 ± 20.09 | 12.91 ± 1.05 | 15.74 ± 1.91 |
| Group5 | 10.32 ± 1.76 | 9.33 ± 4.13 | 7.45 ± 2.72 | 146.34 ± 48.35 | 121.48 ± 31.66 | 14.25 ± 1.26 | 16.93 ± 1.65 |
| Group6 | 23.78 ± 18.04 | 10.67 ± 3.27 | 7.43 ± 3.47 | 156.44 ± 68.53 | 198.48 ± 94.40 | 14.86 ± 2.32 | 33.07 ± 20.46 |
| Group7 | 9.29 ± 1.97 | 6.67 ± 4.13 | 9.15 ± 6.48 | 171.45 ± 99.13 | 186.18 ± 107.93 | 14.21 ± 1.18 | 16.70 ± 1.68 |

Toxicological Study:

At 24 hours of celastrol treatment, blood was collected from the rats in plasma kinetic study. Serum ALT, AST, creatinine, and urea levels are shown in Table 4.

TABLE 4
Serum chemistry parameters in rats after a single oral dose of celastrol (1.8 mg/kg)
with and without different dose of Schisandrin A. Data are the mean ± S.D. (n = 6).

| | Group 1 | Group 2 | Group 3 | Group 4 | Group 5 | Group 6 | Group 7 |
|---|---|---|---|---|---|---|---|
| ALT | 54.5 ± 6.7 | 51.2 ± 3.5 | 54.5 ± 5.7 | 52.5 ± 5.8 | 49.3 ± 6.1 | 52.4 ± 8.3 | 53.4 ± 7.1 |
| (U/L) | | | | | | | |
| AST | 100.0 ± 7.0 | 92.0 ± 6.0 | 91.0 ± 4.0 | 91.0 ± 9.0 | 106.0 ± 14.0 | 111.0 ± 40.0 | 92.0 ± 9.0 |
| (U/L) | | | | | | | |
| Creatinine | 0.48 ± 0.01 | 0.49 ± 0.05 | 0.48 ± 0.02 | 0.49 ± 0.02 | 0.50 ± 0.02 | 0.49 ± 0.01 | 0.49 ± 0.03 |
| (mg/dL) | | | | | | | |
| Urea | 19.6 ± 2.7 | 20.1 ± 1.8 | 19.4 ± 2.5 | 18.3 ± 1.5 | 16.5 ± 1.5 | 17.9 ± 4.0 | 18.8 ± 3.4 |
| (mg/dL) | | | | | | | |

### Example 3

Animal Treatments:

42 Male Sprague Dawley rats (weight, 170-210 g) were maintained on a 12-h light/dark cycle with free access to water and lab chow for 10-14 h prior to the experiment. Rats were randomly divided into 7 groups (n=6 each) and groups 2-7 received 0 mg/kg, 6.3 mg/kg, and 12.6 mg/kg, 25.2 mg/kg, 50.4 mg/kg and 63.0 mg/kg of Schisandrin A in 1% CMC-Na (300-800 cps) via oral administration, respectively. After 5 minutes, all the groups received 3.0 mg/kg of colchicine in a solution of 2% DMSO-98% sterile water via oral administration.

Kinetic Study:

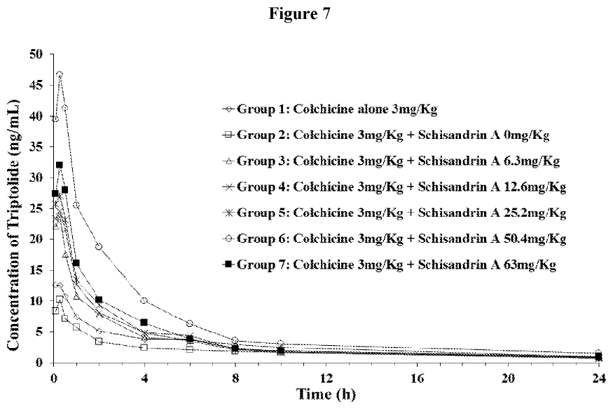

After colchicine treatment, blood samples were collected from individual rat at 5 min, 15 min, 0.5 h, 1 h, 2 h, 4 h, 6 h, 8 h, 10 h, 24 h respectively. Plasma was separated by centrifugation at 8000 rpm for 6 minutes at 4° C. and kept at −80° C. until analysis. Plasma homogenate was injected into LC-MS/MS for analysis. Plasma colchicine concentration-time profiles in different group were shown in FIG. 7. Pharmacokinetic parameters of colchicine after a single oral dose of colchicine (3.0 mg/kg) in rats with and without different dose of Schisandrin A, including area under the concentration-time curve (AUC), mean residence time (MRT) and terminal elimination half-life (T_{1/2}), Cₘₐₓ, and Tₘₐₓ are shown in Table 5.

TABLE 5
Pharmacokinetic parameters of colchicine after a single oral dose of colchicine (3.0 mg/kg)
in rats with and without different dose of Schisandrin A. Data are the mean ± S.D. (n = 6).

| | Pharmacokinetic parameters of colchicine | | | | | | |
|---|---|---|---|---|---|---|---|
| | T_{1/2} | Tₘₐₓ | Cₘₐₓ | AUC_{0→t} | AUC_{0→∞} | MRT_{0→t} | MRT_{0→∞} |
| Group | hr | hr | ng/mL | hr*ng/mL | hr*ng/mL | hr | hr |
| Group1 | 5.20 ± 2.31 | 0.17 ± 0.09 | 13.41 ± 4.38 | 48.91 ± 14.94 | 57.46 ± 16.10 | 4.54 ± 1.33 | 6.99 ± 1.99 |
| Group2 | 4.67 ± 2.04 | 0.22 ± 0.07 | 10.25 ± 2.63 | 32.91 ± 20.90 | 40.58 ± 22.17 | 3.89 ± 1.74 | 6.86 ± 2.77 |
| Group3 | 6.12 ± 2.86 | 0.19 ± 0.09 | 24.36 ± 6.12 | 74.41 ± 26.96 | 84.50 ± 30.54 | 5.04 ± 1.86 | 7.67 ± 3.34 |
| Group4 | 6.56 ± 4.70 | 0.19 ± 0.09 | 25.73 ± 9.67 | 70.99 ± 33.30 | 79.66 ± 34.85 | 3.95 ± 1.20 | 6.54 ± 3.18 |
| Group5 | 7.53 ± 6.49 | 0.11 ± 0.07 | 29.32 ± 11.90 | 75.51 ± 27.60 | 86.79 ± 36.24 | 4.04 ± 1.44 | 7.29 ± 4.72 |
| Group6 | 13.05 ± 6.29 | 0.31 ± 0.16 | 48.22 ± 12.43 | 151.01 ± 41.43 | 185.66 ± 70.47 | 5.10 ± 0.77 | 11.72 ± 6.87 |
| Group7 | 7.59 ± 3.65 | 0.26 ± 0.13 | 33.83 ± 14.04 | 84.76 ± 39.27 | 98.71 ± 41.92 | 4.09 ± 1.76 | 7.52 ± 3.62 |

### EQUIVALENTS

It is to be understood that while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement, and variation of the embodiments therein herein disclosed may be resorted to by those skilled in the art, and that such modifications, improvements, and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A pharmaceutical composition comprising a Schisandra sphenanthera extract, a plant-based compound, and a pharmaceutically acceptable carrier.

2. The pharmaceutical composition of claim 1, wherein the Schisandra sphenanthera extract comprises one or more of Schisandrin A, Schisandrin B, Schisandrin C, Schizandrol A, Schizandrol B, and Schisantherin A.

3. (canceled)

4. The pharmaceutical composition of claim 1, wherein the dosage of the Schisandra sphenanthera extract in the pharmaceutical composition is from about 0.1 mg/kg to about 100 mg/kg, from about 0.5 mg/kg to about 75 mg/kg, from about 1 mg/kg to about 50 mg/kg, from about 2 mg/kg to about 20 mg/kg, from about 2 mg/kg to about 15 mg/kg, or from about 4 mg/kg to about 10 mg/kg.

5. The pharmaceutical composition of claim 4, wherein the dosage of Schisandrin A in the pharmaceutical composition is from about 2 mg/kg to about 15 mg/kg.

6. The pharmaceutical composition of claim 1, wherein the plant-based compound comprises one or more of triptolide, colchicine, wilforlide A, celastrol, and their derivatives or analogs.

7. The pharmaceutical composition of claim 6, wherein the triptolide analog comprises one or more of 16-hydroxy-triptolide, triptonide, and tripdiolide.

8. The pharmaceutical composition of claim 1, wherein the dosage of the plant-based compound in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg.

9. The pharmaceutical composition of claim 6, wherein the dosage of triptolide, colchicine, or celastrol in the pharmaceutical composition is from about 0.01 mg/kg to about 100 mg/kg, from about 0.02 mg/kg to about 50 mg/kg, from about 0.05 mg/kg to about 30 mg/kg, from about 0.1 mg/kg to about 20 mg/kg, from about 0.2 mg/kg to about 10 mg/kg, from about 0.2 mg/kg to about 5 mg/kg, or from about 0.3 mg/kg to about 1 mg/kg.

10.-14. (canceled)

15. The pharmaceutical composition of claim 1, further comprising a CYP enzyme inhibitor.

16. The pharmaceutical composition of claim 15, wherein the CYP enzyme inhibitor is a CYP3A inhibitor.

17. The pharmaceutical composition of claim 16, wherein the CYP3A inhibitor comprises one or more of ketoconazole, itraconazole, fluconazole, cimetidine, clarithromycin, erythromycin, troleandomycin, and grapefruit juice.

18. The pharmaceutical composition of claim 1, further comprising a P-glycoprotein inhibitor.

19. The pharmaceutical composition of claim 1, wherein the pharmaceutical composition is in the form of an oral suspension, an aqueous solution, emulsion, a tablet, a spray, or a capsule, a lotion, a gel, or a foam.

20. The pharmaceutical composition of claim 1, wherein the pharmaceutical composition further comprises one or more of a binding agent, a flavor agent, a lubricating agent, a flow agent, a disintegration agent, a delay agent, an organic solvent, a suspending agent, an isotonicity agent, a buffer, an emulsifier, a stabilizer, and a preservative.

21.-27. (canceled)

28. The pharmaceutical composition of claim 20, wherein the concentration of the organic solvent is 0.1% to about 35%.

29.-35. (canceled)

36. The pharmaceutical composition of claim 1, wherein the pharmaceutical composition is suitable for administration intravenously, subcutaneously, intraperitoneally, intramuscularly or intranasally.

37. The pharmaceutical composition of claim 1, wherein the mass ratio of the Schisandra sphenanthera extract to the plant-based compound is at least 3:1, 6:1, 12:1, 24:1, or 30:1.

38. The pharmaceutical composition of claim 2, wherein the mass ratio of Schisandrin A to the plant-based compound is at least 3:1, 6:1, 12:1, 24:1, or 30:1, wherein the plant-based compound comprises one or more of triptolide, colchicine, and celastrol.

39. A method of increasing bioavailability of a plant-based compound in a subject comprising the step of administering to the subject a therapeutically effective amount of a pharmaceutical composition comprising a Schisandra sphenanthera extract.

40.-56. (canceled)

57. A method of treating and/or preventing a disease in a subject, comprising administering to a subject a therapeutically effective amount of a Schisandra sphenanthera extract, a plant-based compound, and a pharmaceutically acceptable carrier.

58.-74. (canceled)
